(19) United States
(12) Patent Application Publication
(10) Pub. No.: US 2004/0127428 A1
(43) Pub. Date: Jul. 1, 2004

(54) Use of D-ribose, including as a topical vehicle, to promote faster healing, including from surgical procedures

(76) Inventor: Keith E. Kenyon, Van Nuys, CA (US)

Correspondence Address: Keith E. Kenyon, M.D., Suite C, 14435 Hamlin Street, Van Nuys, CA, 91401-6205, US

(21) Appl. No.: 10/330,566

(22) Filed: Dec. 28, 2002

(51) Int. Cl. A61K 31/70

(52) U.S. Cl. 514/23

(57) ABSTRACT

This invention discloses the unique ability of D-ribose to act both as a topical carrier and vehicle for topical drugs and a systemic precursor for nucleosides and nucleotides so that it can be used both systemically and topically in conjunction with outside energy devices such as TENS devices, lasers, and other radiation devices, to facilitate healing as well as facilitating anti-inflammatory, anti-infective and anti-neoplastic pursuits in the treatment of injuries and diseases.

### FIELD OF THE INVENTION

This invention relates to the field of enabling both faster systemic and local healing from injuries and pathology, including injuries inflicted by the use of therapeutic devices employing outside sources of energy.

### RELATED APPLICATIONS

This patent application is related to patent application No. 09/545,121, “The combination of non-living-source physical energy and living-source chemical energy to maximize the salvage of ATP” and patent application No. 10/238,064, “Using D-ribose with or without anti-microbial agents to enhance healing and subsequent recovery by both synthesizing and sparing NAD derivatives”.

### BACKGROUND OF THE INVENTION

This disclosure is concerned with treating injuries to, and pathology of, the skin, subcutaneous, and internal structures, including during, and as a result of, surgery. Injuries usually come from trauma, and sometimes that trauma is deliberate as in contact athletics and by iatrogenic means for therapeutic purposes. At other times it is the result of unexpected circumstances, but all injuries heal the same way. Iatrogenic injuries to the skin that result from outside energy modified for use by various transducers, can be burns, sometimes purposeful as with lasers. On the other hand, there is at least one type of therapeutic device that converts outside energy for healing and pain-relief. While these devices are all old art, the use of D-ribose to potentiate healing, inflicted by them or by non-iatrogenic means is not. Although D-ribose has been used topically along with the other nutrient precursors for adenosine triphosphate (ATP), a major role that D-ribose plays has not been addressed, and this disclosure seeks to remedy that by offering a new use for topical D-ribose even when injury is not the only factor, First, we will discuss the use of non-living source energy devices for therapeutic purposes.

There are several types of energy therapeutic devices. They include those that provide various kinds of beta particles (electrons) to the body which are usually electrons in the form of electric currents, those that apply other kinds of radiation including alpha, gamma, X rays, the photons of these having varying wavelengths, as well as devices that directly supply low or high temperatures to a target area. Heat is the end product of all radiation, and some devices employ radiation to produce heat, at times for a surgical purpose and at other times for a soothing purpose. With others the radiation or the electromagnetic particles are presented in the form of low energy streams or currents, which particles themselves are the effective end product, not the heat they may produce.

The prime example of these are electrotherapeutic TENS (transcutaneous electrical neural [or nerve] stimulating) devices described below, so some of these devices are destructive of tissue and some supportive. Therefore, microcurrent (less than one milliampere) TENS devices are not destructive of tissue, while laser, cauterization, and infrared coagulation devices are, as are high-energy alpha, beta, gamma and X-rays. Since microcurrent TENS devices are not destructive of tissue but can be used by themselves to promote healing, we will next discuss their background in order to understand their special relation to those molecules the nutrient D-ribose is a precursor for, vital in the healing process.

With respect to electric currents, man-made electrotherapeutic devices go back to the invention of the electrostatic generator in the 18^{th} century, its use for pain relief being proposed by Benjamin Franklin, but the modern concept of electrotherapeutics was conceived after Franklin's death, shortly after the battery was discovered by Volta and the generator by Faraday. By 1883, the first formal text, “Handbook of Electrotherapeutics”, written by Professor Wilhelm Erb, of the University of Leipzig, was translated into English by L. Putzel, M.D. and published in New York. The technology was quickly abused by charlatans, and it fell into disrepute by orthodox doctors until the 1960's when the gate theory of pain was proposed, and treatment using electrical currents in devices called TENS devices appeared to have some efficacy with the current being much more controlled than with the 19^{th} century devices. This name has stuck even though in this disclosure, with the use of very low amperage, a nerve being stimulated is not as much a target as are individual cells. With higher amperage, nerves are stimulated more directly, and excessive heat is an unwanted byproduct.

In 1981 this inventor appeared on the television program, “That's Incredible” demonstrating how effective a waveform resembling the natural H-reflex waveform was. At about the same time, low amperage versions of chopped DC appeared with such devices as the Alpha Stim unit that concentrated mostly on stimulating the ear acupuncture points with microamperes. Nevertheless, it was believed by most at that time, as did the original investigators such as Erb, that one must feel the current for it to be effective. Ultimately it was proposed that endorphins, natural opiate-like chemicals, were released, enabling the pain to be relieved.

In recent years more attention is being paid to extracellular nucleotide ATP (adenosine triphosphate) in the pain-mitigating and healing process of injuries. As early as 1982 N. Cheng, et al reported in “Orthopedic Surgery” that electric currents could increase tissue ATP in rats. D. H. van Papendorf, et al from the University of Pretoria, South Africa, reported in 2002 the hypothesis that DC micro electric currents (below one milliamp), “significantly generate, release and drive ATP extracellularly which is degraded to adenosine which in turn is responsible for the pain relief with ATP therapy . . . further contributing to the pain relief is the growth-promoting activity by several experimental observations as well as the reduction in inflammation, oedema, swelling (thus accelerating) wound healing etc.”

TENS devices were part of patent application No. 09/545,121 as one of the non-living source energy devices and how they relate to D-ribose. Shortly before the turn of the 21^{st} century, an important ATP precursor, D-ribose, became commercially available at prices low enough to enable it to be mass marketed. Before this Carniglia, et al, in U.S. Pat. Nos. 4,923,851 and 5,391,550 taught that ribose along with other nutrients helped heal induced skin lesions in mice but only as one of several listed precursors. This inventor, as disclosed in continuing-in-part patent application No. 10/238,064, has found that ribose by itself, either taken internally and/or placed on the skin, helps healing significantly and potentiates anti-infective agents when used concomitantly. The reason for this is that ribose is not available in quantity in food like other nutrients are. This component of ATP must be synthesized from glucose in the mitochondria in the glucose hexophosphate shunt (also called the pentose phosphate pathway) where a carbon atom is removed tediously by enzymatic action from glucose and phosphorylated over from 72 to 96 hours to provide the phosphorylated ribose radical. Its use in the pentose phosphate pathway, apart from a separate new use as a topical vehicle, follows in a simplified form, the italics being the tedious, time-consuming part of the pathway needed to remove a carbon atom from glucose: Glucose+protein enzymes and coenzymes (including NAD derivatives)+electrons+phosphate→glucose-6-phosphate+more enzymes+more electrons→6-phosphogluconate+more enzymes and electrons and nucleotide coenzymes through the hexose monophosphate shunt (3-4 days to complete from glucose) to ribulose-5-phosphate→ribose-5-phosphate+(ultimately with) adenine+protein enzymes+enzymes and nucleotide coenzymes+electrons→AM(mono)P+more phosphate AD(di)P→AT(tri)P and energy.

This long reaction not only produces the ultimate energy process, but perpetuates itself by producing the NAD-derivative coenzymes it needs. Once the AMP is formed, cyclic AMP, the hormone messenger, is produced and also by the removal of a phosphate bond, basic adenosine with its myriad uses is formed which plays roles in pain relief, healing and regulating electrical conduction.

On the other hand, if the pentose phosphate shunt can be shortcut, much time is saved by the reaction cutting in after the italics shown above as follows; D-ribose+enzymes and coenzymes (including nucleotides)+electrons+phosphate ribose-5-phosphate+(ultimately with) adenine+protein enzymes+enzymes and nucleotide coenzymes+electrons AMP+more phosphate→ADP→ATP and energy.

Since free ribose is not ordinarily available in food as glucose is, when an injury occurs, the immune system immediately calls upon the mitochondria through the hexose monophosphate shunt to provide the ribose radical for ATP, but ATP is always in high demand by all the other cells in the body even to the exclusion of the production of nucleoside NAD (nicotinamide adenine dinucleotide), key to providing co-enzymatic action for the production of the cloaking protein-protection for our genes. This problem of supply and demand also applies to the nucleotide leukocyte NADPH (reduced nicotinamide adenine dinucelotide phosphate) oxidase to form superoxide via 2 O₂+NADPH→2 O₂⁻+NADP⁺+H⁺ (the symbol “O₂⁻” being superoxide). Superoxide is the body's own first line antibiotic, anti-microbial (i.e. anti-infective) agent. Both NAD and NADPH contain two of these tedious-to-synthesize ribose radicals, and immediately available ribose radicals are always in short supply, when they are needed rapidly. Therefore, they are not always available in sufficient amounts for maximum rapidity in healing unless de novo D-ribose is made available directly, either by ingestion, infusion or topically or by more than one of these routes simultaneously.

As disclosed in patent application No. 09/545,121, the use of such electromagnetic energy discussed above is made significantly better with respect to healing, including from surgical procedures, when D-ribose is taken internally also. As disclosed, other forms of energy, including direct application heat, work better and aid pain relief and healing also when they are employed with D-ribose being taken at the same time, as well as before and after. Of special merit for the use of this disclosure are lasers and infrared coagulation devices, because they result in the destruction of tissue most often on the skin, mucous membranes or eye tissue, the harmful effects of which trauma can be minimized and healed faster by the method of this disclosure, as it can for internal injuries when using D-ribose.

With the above background in mind, we will disclose an important second way D-ribose acts, apart from being an essential, tedious-to-synthesize precursor for AMP, ATP, and the NAD derivatives. While the first is biochemical, the second is biophysical, the carrying of medicaments or drugs with it across the skin, the tunica mucosa, and the cell membranes.

This application seeks to overcome the deficiencies of previous applications and inventions by the use of de novo D-ribose in combination with therapeutic energy devices and topical medicaments, to repair injuries and relieve pain more effectively with greater rapidity and to improve the healing process by a greater identification of the means employed to combine de novo D-ribose with therapeutic energy devices both during surgical and non-surgical procedures, for accidental injuries, and when any medicament is needed to be transported through the skin by a non-toxic, non-drug vehicle.

### BRIEF SUMMARY OF THE INVENTION

When there is either an iatrogenic injury, including a surgical incision or cauterization, or a non-iatrogenic injury, the same mechanism of healing takes place. Platelets automatically attempt to control bleeding when it is present. The immune system sends in repair cells, starting with leukocytes. Inert fibrocytes are converted to fibroblasts. Also myofibroblasts, thought to enable wound contraction, permeate the area, and when bone is involved osteoblasts are proliferated. All of this anti-inflammatory activity depends on cellular energy plus the availability of natural anti-infective agents, the most important one being superoxide, made by leukocytes via leukocyte NADPH oxidase, especially from neutrophils. As disclosed above, extracellular ATP which first comes from the mitochondrial synthesis in the pentose phosphate pathway a.k.a. the glucose monophosphate shunt, plays a role in healing and pain relief as well as its precursor AMP providing NADPH oxidase for the respiratory burst in leukocytes to produce extra superoxide to destroy bacteria and other infective agents.

Low voltage, microamperage (less than one milliamp) electromagnetic energy in the form of micro electric currents increases extracellular ATP and if accompanied by de novo D-ribose in the area, facilitates local NADPH synthesis, both of which molecules are synthesized from nucleotide AMP (adenosine monophosphate) after a carbon atom is removed from glucose in the pentose phosphate pathway. Once ribose has been synthesized into AMP, NAD can be formed, which has two ribose radicals and is the basic coenzyme that is used for enzymatic processes that just require the hydrogen bond. When the phosphorous bond is needed for energy transfer, it is included as NADP. AMP is the first of the final target molecules after the pentose phosphate pathway, each with its own physiological purposes. Shortcutting the pentose phosphate pathway by inserting de novo D-ribose in order to make AMP much faster and then the others, also shortcuts the healing process after the originally available ATP and NAD and its derivatives have been used up and their replacement slowly started.

To help further, simple sugars such as lactose have a capacity to biophysically carry medicaments with them across mucous membranes as well as the skin. This use has long been exploited in homeopathy by the use of lactose as such a carrier. Up until this disclosure this has not been attributed to ribose. Ribose was not available as a research substance until the 1960's and as a cost-effective commercial product only recently.

Unfortunately, D-ribose is still quite expensive, and since no one skilled in the commercial art would think of employing a very expensive substance as a carrier, more costly than the medicaments in many cases, this vehicle use has not been considered. Lactose, on the other hand, being a milk product, has had low cost for centuries. Nevertheless, D-ribose being the essential sugar it is, has no trouble being absorbed into the body. This inventor, being a physician, has discovered that it carries anti-infective agents across injury sites, including burns with such speed that the antibiotic action takes place much faster, even immediately, and continues its acceleration during the entire healing process, starting much before the second or precursor action kicks in after about eight hours. Thus, D-ribose has a carrier action both with respect to its use as a solid carrier or vehicle and an iontophoretic carrier.

When it comes to the use of D-ribose as an iontophoretic carrier, so associated with microcurrents, the potentiation is remarkable for a number of protocols including the following protocol using D-ribose in an aqueous solution, which is impregnated into a sponge-type electrode. Whereas, with respect to closed injuries on the body itself (or just plain internal pain from arthritis, tendonitis, toothaches or other nerve pain), the electrodes after at least one is made wet with aqueous D-ribose to promote a number of actions, including extracellular ATP being both released and synthesized faster, the wet electrode can be placed over the injury site with a dry but conductive one elsewhere on the body. The ribose solution, whether used on one or more electrodes, can be of various strengths, even saturated, but from 2% to 5% works well and is cost-effective. Low strengths down to 0.01% and even lower can be marginally effective, but higher strengths are preferred. When two electrodes are wet (although for multiple injuries, multiple electrodes can be used with one unit if the average amperage is sufficient in strength to accommodate all), they should both be placed near the injury with care taken to make sure they do not touch each other. When the injury is such that it would be awkward to make sure the electrodes don't touch, a non-ribose second electrode can be placed remotely or with short-term applications a copper connection or “ground” can be held in the hand. An ECG electrode can be used as a second electrode, but increased amperage may be needed over two wet ones. When the wound is vaginal or rectal (usually from surgery) an electrode with ribose can be inserted intra-orifice while the second one (not necessarily needing to have ribose but can even be an ECG electrode) is placed outside the orifice. The unit being quite small can be taped to the buttocks or waist. The wet sponge-type electrode can be removed and a new one with D-ribose inserted as needed. Later the vehicle or carrier use of D-ribose in topical antibiotic and other drug formulations will be discussed.

This procedure is being disclosed in two ways. The first is when the injury is anticipated. Then administer D-ribose systemically in advance to enhance more rapid healing when the surgical instrument uses energy to therapeutically cause the injury. While many iatrogenic injuries are caused by an obvious injury-creating entity such as a laser, electrocautery or infrared coagulator, including the Redfield IRC 2100, or other similar heat-producing instruments, some produce only a tiny amount of injury such as for ophthalmologic uses and removal of a clot in a coronary artery. Some of these tiny iatrogenic injuries occur in stent procedures in the coronary arteries to remedy occlusions. The fact that healing is not always successful, resulting in re-stenosis, emphasizes the need for the healing ability D-ribose in the blood stream offers. In these cases, administering oral or parenteral D-ribose, before, during, and after the procedure reduces the chances for poor healing, which in the case of stents can be life-saving. This is done by employing D-ribose most commonly by mouth, but for surgical procedures where possible, administered by intravenous infusion, starting for a period of time usually at least eight hours prior to the surgical procedure, but shorter when intravenous application is used On the other hand, the D-ribose can start to be administered at any time following surgery with a lower, but still considerable degree of success. When the injury is close to the surface after the procedure, D-ribose can be employed topically with drugs, medicaments or pharmaceutical agents listed in the Physician's Desk Reference, in ointment, gel, cream, or lotion with or without a microsphere vehicle.

The second way is when an injury is unexpected and not caused by doctors, so ribose would not ordinarily be taken prior. Once an injury or pathological condition has occurred, D-ribose can be used topically over the targeted site. This can be done using a solution of the D-ribose alone, or on the electrodes conveying a micro electric current, or as an ointment, gel, cream, or lotion with anti-microbial agents and even other agents as disclosed below, taking advantage of D-ribose's use as a carrier of such substances. As part of a topical vehicle, ribose's soothing nature enhances quality of life.

With respect to rectal hemorrhoids, D-ribose can be used with all techniques including using the rubber band technique or used in combination with a scalpel after tying off a severe hemorrhoid. Also an infrared coagulator, preferably the Redfield IRC 2100, can be used for less severe pathology. Both types of procedures are improved with parenteral or local D-ribose or both, with local D-ribose also being valuable when accompanying micro electric currents from an appropriate very low amperage therapeutic electromedical device or as it frequently would be called with old terminology, a very low amperage TENS device.

### ADVANTAGES OF THIS INVENTION

1. It promotes faster healing of open wounds whether surgical or traumatic.

2. It produces greater pain relief from open wounds.

3. It produces greater pain relief from closed injuries or painful pathological conditions by releasing more extracellular ATP and faster.

4. The transmitting device can be made so small that it can be worn comfortably on the body.

17. In the event that the individual is squeamish about even a very low amperage unit that imparts no sensation, so does not want to insert electrodes or have a small electromedical unit attached, by giving ribose orally in advance before, during and after the surgery, potentiation of healing is still obtained.

6. The oral dose of D-ribose can be given dissolved in water so the daily dose of at least one gram but preferably 10 to 20 grams can be varied easily to suit the patient in need of healing.

7. D-ribose can be provided with anti-infective agents to potentiate them.

8. D-ribose can be used as a topical vehicle or carrier of medicaments across the skin, subcutaneous tissues and cell membranes.

9. D-ribose can be used internally or parentally to protect coronary stents from failing to heal fast enough to prevent re-stenosis.

The features of the present invention which are believed to be novel are set forth with particularity in the appended claims. The present invention, both as to its organization and the manner of operation, together with the further objects and advantages thereof, may be best understood by reference to the following exemplary and non-limiting detailed description of the invention, wherein;

### DETAILED DESCRIPTION OF THE INVENTION

The present invention comprises a method employing a series of steps to use D-ribose in the various ways its unique structure accomnodates, including as a carrier and as a topical vehicle of medicaments by itself, as a precursor for ATP, AMP and NAD, and finally in combination with a variety of therapeutic devices that employ non-living source energy such as obtained directly from an electric generator or batteries, by high temperature or refrigeration means or by various radiation devices, including X rays and radioactive material. While the method of this disclosure is primarily planned to be used on human beings, it can be used on animals also because it is a treatment procedure involving living, or derived from living, tissue that has been injured or needs topical therapy. The method comprises steps that involve employing or enduring said non-living source energy in combination with D-ribose, an essential precursor for nucleotides AMP, ATP and NADPH and nucleoside NAD and as a carrier and topical vehicle for medicaments across normal or injured skin. D-ribose as a synthesized precursor is not ordinarily immediately available from food, because, its synthesis is by a tedious, lengthy process, however, made much shorter by supplying de novo D-ribose.

On the other hand, as a carrier of medicaments it is immediately available for the action of transporting medicaments across a barrier such as the skin or mucous membranes and then with a delayed action, but much shorter than the full hexose monophosphate shunt, acting as a precursor, so its application can have a combined action, the first being as a vehicle.

With the infliction of energy-caused trauma resulting in injuries, with or without a surgical or therapeutic purpose, D-ribose can be given orally, mixed with other substances, including anti-microbials, cortical steroids, and certain anti-neoplastic drugs, by intravenous infusion, by placement into the rectum and even inside the abdomen during surgery. It also can be applied topically to the skin with other medicaments, especially anti-microbials and topical anti-neoplastics, the ribose acting as a vehicle and carrier for these as well as its own precursor use. It can be administered as a solid by mouth or as an aqueous solution and, of course, parentally in the form of an intravenous aqueous solution similar to the various kinds that employ glucose and water.

Non-living-energy required for therapeutic units, including lasers, cauterization devices, infrared coagulators, skin penetrating scopes like arthroscopes, electrotherapeutic (TENS) devices, cryogenic devices, high-energy radiation devices and materials, ultrasonic devices, vibratory devices and simple heat and ice packs are well known and commercially available.

This disclosure is designed to be of use in part when such devices cause damage to the living tissue they are dealing with as part of their purpose. Thus a cryogenic device used to remove a shallow skin lesion actually freezes the pathological tissue and some surrounding tissue resulting in the same kind of damage as if a burn occurred. A laser actually burns a tiny amount of tissue with each application to realize its objective. An infrared coagulator burns a larger amount of tissue with a single application than does the laser, because it cannot be so precisely focused as the laser, even though photons are the particles delivered in both cases, just with different wave lengths. In surgery an electro-cauterization device imposes burns also. In addition toxic anti-neoplastic drugs irritate the skin when used to treat pre-cancerous skin lesions.

This disclosure further seeks to offer means to enable faster healing, less irritation, with minimal scarring by using D-ribose in various ways in the therapeutic procedure. When de novo D-ribose is given, a considerable amount of the pentose phosphate pathway is eliminated, saving as much as 88 hours of the up to 96 hours the entire pathway takes at its longest. Even if it takes only the minimum of time, 72 hours, 64 hours are saved. All the other precursors of ATP and NADPH are immediately available in food, but if de novo D-ribose is administered into body orifices, including the mouth, ears, nose, rectum, or vagina or placed on various locations on the skin by itself or preferably on the skin with anti-infective agents, besides its carrier use, the other precursors of ATP and NADPH, all available immediately from food, can be utilized by the body faster as long as the D-ribose is there already, to have all of the ingredients quickly available to increase the amount of these vital molecules.

Whereas, electricity in the hands of a surgeon can be destructive as in the case of the electro-cautery during the surgical procedure, special note in this disclosure is for the use of electric currents when they are not destructive but supportive. This occurs after the surgery when such currents can be used at the wound site to effect faster healing. In order to be completely safe and work best, these currents must be less than one milliamp, and preferably less than 12 volts.

The electrodes can be of various numbered pairs, usually two or four electrodes, and can be the kind of electrodes used to monitor the heart such as those used for ECG's when only parenteral D-ribose is given. When D-ribose in solution is placed on electrodes for transcutaneous delivery, they can be of sponge material with liquids soaked in. The cotton tip of an applicator stick can also be used. The liquids can be ordinary tap water or saline solution or have special electrically conducting material in them. In this disclosure sponge-type electrodes, cotton applicator stick tips, or even bandages soaked in an aqueous solution can be used, but in addition to any other ingredient or without any other ingredient, D-ribose dissolved in the water in various strength solutions is of primary importance, and a major reason for this disclosure. As long as D-ribose is in one sponge-type electrode, the other electrode can be an ECG or equivalent electrode or a hand held conductor of copper or other highly conductive material.

When used as a topical preparation without the soothing and healing help of a TENS device, besides being active itself from the precursor point of view, as exemplified by the Carniglia, et al patents discussed in the Background, and as all the prior art involved with D-ribose been restricted to, its action as a physical carrier for other substances as part of a topical vehicle has never been disclosed. This omission being addressed here includes using D-ribose as a carrier for anti-microbial agents, vaginal and rectal agents, corticosteroids, anti-neoplastic and other drugs that when water soluble can be in a solution with D-ribose but usually as a solid mixture containing D-ribose as part of the vehicle and with one or more medicaments, even toxic ones like 5-fluorouracil. It can be used without an electric current in aqueous solutions alone for this purpose, but mostly it is used in ointments, gels, creams, and lotions as a vehicle or part of a vehicle. The mechanism whereby DMSO penetrates the skin and carries therapeutic agents through it may differ from that of ribose, since DMSO is a solvent and ribose and sugars like it such as lactose are solids. Nevertheless, D-ribose especially has the capacity to cross membranes, being a key ingredient in the molecule cells want most, ATP, and experience has taught us that when a wound or bum is encountered, ribose and antibiotics work faster than antibiotics alone, so ribose must make them more available. This, of course, can extend to other medicaments, including 5-fluorouracil and certain refined herbs or herb-derived chemicals. When the electric current is used, the ingredients are also transported by iontophoresis, including the ribose, and then the ribose in turn may carry even higher concentrations of the other ingredients with it across the skin and then across target cell membranes. The medicament can be more localized with microspheres, and the ribose can incorporated with or into microspheres.

The use of certain sugars as carriers of other substances has a long history. Samuel Hahnemann who founded the healing science, homeopathy, found that lactose could be used as a carrier of substances rendered homeopatbic. Ethanol and even DMSO, being liquid, are more convenient to use as topical carriers, but D-ribose dissolved in water is also effective with a multiple action as disclosed in this application. When solids are involved, as is the case with antibiotic topical preparations, D-ribose can again be a very effective carrier as is homeopathic-used lactose as a solid. When D-ribose is used in a topical preparation, the ribose acts in several different ways, including like the carrier sugar of homeopathic lactose, as a soothing part of a topical vehicle, and as its own unique use as a hard-to-get-by-food precursor of key elements in the healing process.

When D-ribose is used in place of lactose for homeopathic medicaments, the medicaments remain the same as in standard homeopathic preparations, however, when microcurrent electromedical electrodes are used also, the energy of the electrical current is additive to the energy of the homeopathic-type preparation with the D-ribose.

When D-ribose is used solely as a topical vehicle for carrier and vehicle purposes, many ingredients such as anti-itch creams, gels, ointments, or lotions, including commonly used ones such as diphenhydramine and calamine lotion, can be combined with it to facilitate absorption and increase the effectiveness. Since D-ribose is an immediate precursor to important coenzymes and energy molecules, it has both a therapeutic use of itself and a carrier use to facilitate the absorption of the accompanying ingredients, whatever they are. When D-ribose is used as one part of five, it has become 20% of the formulation, which is practical for over-the-counter preparations, although even higher concentrations can be used. Lower concentrations can be used also with considerable effectiveness, especially if the non-ribose ingredients are inexpensive and low cost is vital. On the other hand, when the non-ribose ingredients are much more expensive than the ribose, a greater proportion of ribose used can be more cost effective, because it can carry a larger proportion of expensive ingredients by its abundance. Ribose has a soothing quality to it and it imparts this soothing ability to ingredients that accompany it, even if they are soothing also.

Of special interest with respect to using D-ribose with energy therapy devices is the use of infrared coagulation, which like the laser provides therapeutic light, which quickly becomes heat Of the two, the infrared coagulators provide more injury per application than the lasers. This is because the laser is much more precise in the amount of injury it inflicts as part of its surgical removal of a lesion or hair or its knife-like use in the radial keratotomy procedure. D-ribose in any form of delivery can aid the healing of these uses. With the laser there is less tissue destruction than with the infrared coagulator per individual application with the transducer. Therefore, although D-ribose can be beneficial for both, it is even more beneficial for iatrogenic burns using the infrared coagulator in the obliteration of tattoos as an example. Since the infrared coagulator is a much less expensive instrument, treatments can cost less, while the use of D-ribose can make patients less prone to poor healing of the burned area.

While the Redfield IRC 2100 is the best infrared coagulator on the market at the time of this disclosure, other brands can be helped therapeutically in the recovery of the tissue by concomitant use of D-ribose, either topically or internally. Patent application No. 10/238,064 disclosed how anti-microbial agents could be potentiated with D-ribose. In the case of burns from lasers or infrared coagulators, the combination of a topical mixture of antibiotic ointments, and D-ribose can work very well to promote faster healing and decrease infection even without taking it by mouth or by other routes concomitantly, although combining parenteral and topical means works best. Many different amounts of D-ribose compared to antibiotics can be used effectively, but the use of 5 grams of D-ribose mixed with 15 grams of antibiotic ointments is a good mixture. When preferred, gels can be substituted for ointments with 5 grams of D-ribose used per 15 grams of antibiotic gel. Substances like Silvadene are used in the same way. While more ribose can be used, this ratio keeps the texture of the ointment or gel more similar to its texture before adding ribose. The commonest antibiotics used this way are bacitracin, polymixin, and neomycin either together or separately. D-ribose can be added to such topical material and any antibiotic recommended for topical use in the Physicians Desk Reference can be used also. If D-ribose makes an expensive topical antibiotic work better, it is very cost-effective. Of course, being inexpensive for topical use, D-ribose makes any topical preparation it can aid, more cost effective, including the routine combined triple antibiotic ointments and their preparations as solitary antibiotics also.

Once the infection is under control, the minimizing of scarring is desirable. At the present time cortisone preparations, including new ones such as clocortolone pivalate, are the treatment of choice. Nevertheless, D-ribose as described above can be combined with cortisone in a gel, cream or ointment. Once again, although lower concentrations can be used, to go above one gram of ribose per three grams of cortisone ointment, cream or gel, interferes with the uniformity of the product, and greater amounts of D-ribose than these are not needed. The microcurrent electrotherapeutic device with D-ribose solution on the electrodes as described above, further contributes to minimizing scarring and keloid formation. It is important to apply pressure to the scar as part of the healing procedure.

Another use for D-ribose is with certain skin lesions that are the result of solar-caused degeneration of the skin by exposure to the sun. This condition is commonly referred to as actinic keratosis. In the course of his research on himself, this inventor noticed that after several months of the daily intake of 10 grams of D-ribose by mouth, large actinic keratoses on his forehead and wrists simply disappeared. Since 5% of these lesions degenerate into squamous cell carcinomas, the use of ribose alone would be useful. On the other hand, it also may be used as part of a topical formulation. Topical 5-fluorouracil formulations (5-fluorouracil being a pyrimidine analogue with anti-neoplastic action because certain neoplasms incorporate uracil into their ribonucleic acid more readily than in normal cells) have been used to eradicate actinic keratosis in place of “spot” cryosurgery and curettage, with as much as 5% fluorouracil used. Recently there came on the market a new microsphere-based formulation using a microsphere vehicle called Microsponge® in a topical cream containing only 0.5% fluorouracil. It is applied once daily with good results and less than 1/40^{th} the systemic fluorouracil absorption compared to that of 5% fluorouracil. The course of treatment ranged over a 4-week period. While using D-ribose systemically by itself works slowly on presumed, non-malignant actinic keratoses and senile keratoses when malignancy is questioned (as it always must be even with a histological or clinical diagnosis to the contrary), using D-ribose systemically is not enough for every case.

Whether or not systemic D-ribose is used, incorporating it by a series of steps into a topical vehicle for both carrier and anti-inflammatory purposes for the administration of topical drugs (i.e. pharmaceutical agents) listed in the Physician's Desk Reference enables most or all such drugs to gain entry through the skin faster, be less irritating, with faster healing when needed on the site where the preparation is administered. Pharmaceutical 5-fluorouracil, serves as a good example. The 5-fluorouracil topical preparation, with or without the preferable microsphere-based preparation, increases the efficacy over not using the topical D-ribose, with even more potential decrease in toxicity of the 5-fluorouracil. While lower amounts of D-ribose in the topical formulation can be used for economic reasons, 10% or more D-ribose is preferred for carrier and anti-inflammatory purposes. When 20% D-ribose is incorporated into a commercial brand of 0.5% 5-fluorouracil cream (Dermik Laboratories of Berwyn Pennsylvania's, Carac™), it reduces the 5-fluorouracil content to 0.4% with no change in efficacy and with even lower toxicity and side effects, such as site irritability, with faster return to normal skin once treatment has stopped. Continuing the ribose without 5-fluorouracil when the fluorouracil treatment has been discontinued enables relief from irritation to end even sooner rather than the two weeks it now takes. Furthermore, using D-ribose with 5-fluorouracil enables longer treatments to be tolerated on a case-by-case basis. The same scenario applies to other such agents, including 3M Pharmaceutical's imiquimod.

Even for benign conditions like acne vulgaris, D-ribose can act as a carrier and vehicle of medicaments such as tretinoin and benzoyl peroxide, the latter having a number of other uses, including for scalds and burns, but also as a keratolytic in the treatment of acne vulgaris. Benzoyl peroxide has been combined with antibiotics such as erythromycin or clindamycin in a topical preparation. It has a specially noted action against Propionibacterium acnes and when combined with antibiotics, is even more effective. With or without antibiotics, the use of D-ribose as a carrier and vehicle for benzoyl peroxide demonstrates another of the many uses of D-ribose in topical preparations. It is interesting to note that a gluco-based isosorbide derivative is being used as a vehicle for benzoyl peroxide formulations, and D-ribose and it can be used together.

Next, we will disclose the special situation that applies to occlusion of a coronary artery. Energy is imparted to various means to remove the occlusion and when removed, an iatrogenic surgical injury has occurred, requiring the immune system to start the healing process. In this case speed and completeness of healing is vital. Unfortunately, a foreign body (the stent) needs to be placed at the site of healing. The need for ribose's healing help becomes paramount now to make sure enough ATP, AMP, and NAD and its derivatives are available as soon as possible and in optimum amounts. This can best be done if the D-ribose is started at least eight hours prior to surgery.

Finally, while we have used the common term, ribose, in this disclosure, it is not limited to ribose, and includes any 5-carbon precursor of ribose, D-ribose, ribulose, xylitol and xyulose.

While particular variations of the present invention have been described, it will be obvious to those skilled in the art that changes and modifications may be made without departing from my invention in its broader aspects of a method to utilize devices employing non-living energy, such as infrared coagulators and lasers, to remove unwanted or pathological living or derived from living tissue in combination with various dispensing forms of D-ribose, said D-ribose with or without microcurrent electromedical devices to promote faster healing of iatrogenic and non-iatrogenic injuries and pathological conditions. 25

I claim:

1. The method of employing an amount of D-ribose for a therapeutic purpose in a treatment procedure involving injured as well as infected living tissue comprising the steps of:
a. obtaining at least one milligram of D-ribose for the purpose of making it available both inside the body of a living human being or animal containing said tissue, at the injury site of said tissue, or both, and then;
b. preparing said D-ribose to be administered by oral, parenteral or topical means and then;
c. combining said D-ribose with one or more pharmaceutical anti-infective, anti-microbial agents to facilitate the healing activity by limiting infection or treating it when present.

2. The method according to claim 1 in which D-ribose is administered topically to act as a vehicle and biophysical carrier for medicaments and other ingredients in composition with it.

3. The method according to claim 1 in which said D-ribose preparation is combined in the treatment process with a source of outside, non-living physical energy employed in a device capable of placing that energy on or within a human being or animal body.

4. The method according to claim 3 in which said outside physical energy device has a transducer, including one being fashioned in the form of a probe, to place said energy on or in the body in small target areas.

5. The method according to claim 3 in which said device employing non-living source physical energy emits electromagnetic radiation from infrared to gamma rays.

6. The method according to claim 5 in which said infrared rays are used for coagulation.

7. The method according to claim 6 in which said infrared coagulator is a Redfield IRC 2100.

8. The method according to claim 3 in which said device produces an injury inside a coronary artery when removing an occlusion and inserting a stent.

9. The method according to claim 1 in which D-ribose is taken internally during a surgical procedure involving a laproscope or placement of a stent.

10. The method of employing an amount of D-ribose for a therapeutic purpose as a part of a topical vehicle for any drug listed in the Physicians Desk Reference that has a topical application, comprising the steps of:
a. obtaining at least one milligram of D-ribose and then;
b. obtaining at least the minimal therapeutic amount of said drug as listed in the Physicians Desk Reference, and then;
c. mixing them together in varying amounts of each in a topical formulation and then;
d. applying said formulation to one or more areas of the skin.

11. The method according to claim 10 of employing an amount of D-ribose for a therapeutic purpose in a treatment procedure involving solar damaged skin such as actinic keratosis comprising the steps of:
a. obtaining at least one milligram of D-ribose and then;
b. obtaining at least one milligram of 5-fluorouracil and then;
c. mixing them together in varying amounts of each in a topical formulation and then;
d. applying said formulation to areas of the skin affected by solar degeneration such as actinic keratosis and its neoplastic complications.

12. The method according to claim 10 in which said drug or pharmaceutical agent is benzoyl peroxide.

13. The method according to claim 10 in which said drug or pharmaceutical agent is tretenoin.

14. The method according to claim 10 in which a microsphere vehicle is incorporated into said topical preparation.

15. The method according to claim 14 in which Microsponge® is said microsphere vehicle.

16. The method according to claim 11 in which Dermik Laboratories' Carac™ is the formulation into which the D-ribose is incorporated.

17. The method according to claim 11 in which imiquimod is used in place of fluorouracil.

18. The method according to claim 11 in which any anti-actinic keratosis agent is used in place of fluorouracil.

19. The method according to claim 10 in which any drug or pharmaceutical agent for acne vulgaris is used.
